(19) United States
(12) Patent Application Publication
(10) Pub. No.: US 2020/0038248 A1
(43) Pub. Date: Feb. 6, 2020

(54) Negative Pressure Enhanced Cellular Infiltration

(71) Applicant: Temple University- Of the Commonwealth System of Higher Education, Philadelaphia, PA (US)

(72) Inventors: Azadeh Timnak, Philadelphia, PA (US); Jonathan A. Gerstenhaber, Philadelphia, PA (US); Yah-el H. Har-el, Philadelphia, PA (US); Peter I. Lelkes, Cherry Hill, NJ (US)

(21) Appl. No.: 16/339,955

(22) PCT Filed: Oct. 5, 2017
(86) PCT No.: PCT/US17/55273

§ 371 Date: Apr. 5, 2019

Related U.S. Application Data
(60) Provisional application No. 62/404,574, filed on Oct. 5, 2016.

(51) Int. Cl. A61F 13/00; A61M 1/00; A61F 13/36; A61L 15/40; A61L 15/44; A61F 13/15

(52) U.S. Cl. CPC A61F 13/00068; A61M 1/0088; A61F 13/15658; A61L 15/40; A61L 15/44; A61F 13/36

(57) ABSTRACT

The present invention provides methods for improving negative pressure wound therapy. The application of negative pressure to a wound bed with a biodegradable biocompatible fibrous scaffold facilitates cell infiltration into the scaffold serving as a 3D structure, as opposed to solely application of the scaffold without any pressure, which shows minimal cell penetration. In vitro studies show that infiltration of immune cells into scaffolds paves the way for their polarization towards the phenotype pertinent to the remodeling stage 2. The cell penetration factor causes less intense immune response of the host body, leading the inflammation to proceed to the remodeling stage.

### CROSS-REFERENCE TO RELATED APPLICATIONS

This application claims priority to U.S. Provisional Patent Application No. 62/404,574, filed Oct. 5, 2016, the contents of which are incorporated by reference herein in its entirety.

### BACKGROUND OF THE INVENTION

Chronic wound care imposes enormous financial burden to both the U.S. healthcare system and to personal payers. Currently, the annual cost associated with non-healing/hard-to-heal skin wound care adds up to about 25 billion dollars (Huang, C et al., 2014, Current problems in surgery 51(7):301-331). However, in spite of all the costs, the patients' need of living a normal life without any pain or scarring are barely met. Currently, autografting is the gold standard in skin replacements. Nevertheless, due to morbidity, mortality, and limitations in the area of skin available to be transplanted, an alternative treatment is necessary. Currently, treatment modalities such as the simple application of wound dressing or bioengineered skin replacements are available. Application of wound dressings can effectively balance moisture of the wound bed, though scarring and necrosis of the tissues are still highly possible. Application of bioengineered scaffolds in animal models seems to play a role in restoration of skin appendages (Har-el, Y et al., 2014, Wound Medicine 5:9-15; Sundaramurthi, D et al., 2014, Polymer Reviews 54(2):348-376). Reestablishment of the skin appendages represents initiation of the remodeling stage, the final and desired stage of the healing process. Nevertheless, prolonged inflammation is the obstacle hindering remodeling of the healing tissue (Wang, Z et al., 2014, Biomaterials 35(22):5700-5710; Mantovani, A et al., 2013, The Journal of pathology 229(2):176-185; Badylak, S F et al., Tissue Engineering Part A 14(11):1835-1842).

There is a need in the art for improved techniques for healing wounds. The present invention meets this need.

### SUMMARY OF THE INVENTION

The present invention provides a method of enhancing wound healing, comprising the steps of: providing a scaffold; packing a wound with the scaffold; encasing the wound with an airtight cover connected to a vacuum source; and applying at least one instance of negative pressure to the wound using the vacuum source.

In one embodiment, the provided scaffold is pretreated by the steps of: placing a population of cells near the scaffold in a container connected to a vacuum source; and applying at least one instance of negative pressure to the container. In one embodiment, a population of cells is placed near the scaffold packed into the wound.

In one embodiment, the provided scaffold comprises electrospun soy protein isolate. In one embodiment, the provided scaffold is electrospun from a solution comprising 7% (w/v) purified soy protein isolate (SPI) and 0.05% (w/v) polyethylene oxide (PEO) dissolved in 1,1,1,3,3,3-Hexafluoro-2-propanol (HFP). In one embodiment, the provided scaffold comprises fibers having a diameter of between 0.5 and 5 μm.

In one embodiment, the population of cells comprises at least one cell selected from the group consisting of: fibroblasts, keratinocytes, melanocytes, monocytes, and macrophages.

In one embodiment, the wound is a cutaneous wound, a muscle wound, a diabetic ulcer, a burn wound, or a surgical opening.

In one embodiment, the at least one instance of negative pressure is applied using a negative pressure wound therapy system. In one embodiment, the at least one instance of negative pressure applied is between 1 and 10 psi. In one embodiment, the at least one instance of negative pressure is applied for a duration of between 5 and 50 seconds. In one embodiment, the at least one instance of negative pressure is applied continuously. In one embodiment, the at least one instance of negative pressure is applied in a pulsatile pattern.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a flowchart for an exemplary method of negative pressure wound therapy.

FIG. 2 depicts an NPWT system used for the management of chronic or acute wounds. Application of vacuum in vivo helps to remove infectious factors, exudates and irrigation fluids from the wound bed, enhances circulation, and improves recruitment of cells involved in wound healing process.

FIG. 3 depicts an exemplary electrospinning setup. During the electrospinning process, soy protein isolate (SPI) solution is pumped from a syringe pump into a charged field. The polymer is attracted to the aluminum coated target across the charged field, the solvent evaporates in flight, forming nano-fibrous SPI fibers that land on the target and create a non-woven fibrous mat/scaffold.

FIG. 4 depicts a soy scaffold imaged by Scanning Electron Microscopy (SEM) with accelerating voltage of 20 kV at two magnifications.

FIG. 5A depicts a stress-strain curve of the soy samples (5 mm×20 mm) under dry and wet conditions.

FIG. 5B depicts the ultimate tensile strength of the samples in FIG. 5A. The wet samples were soaked in water overnight prior to mechanical testing. On test day the samples were taken out of the water and tested immediately. Statistically significant differences (P<0.01) between dry and wet samples were observed using a t-test.

FIG. 6A and FIG. 6B depict human keratinocyte (HaCaT) cells seeded on top layer of electrospun soy scaffolds (25,000/cm²). Surface view of scaffolds (FIG. 6A) under standard condition and (FIG. 6B) following brief application of negative pressure. Nuclei stained with DAPI are shown in blue and SPI scaffold autofluorescence is shown in green. Images were taken 3 days post seeding using a laser scanning confocal microscope (LSCM).

FIG. 7A and FIG. 7B depict cellular penetration into the mid-layer (70 m in depth) of electrospun SPI scaffolds (FIG. 7A) under standard condition and (FIG. 7B) following application of negative pressure. Blue: nuclei, green: scaffold autofluorescence. Scale bars: 100 μm.

FIG. 8A and FIG. 8B depict 3D-reconstructed laser scanning confocal microscopy (LSCM) images using MATLAB® showing the location/penetration of HaCat cells after seeding on electrospun SPI scaffold with (FIG. 8A) under standard condition; (FIG. 8B) following application of negative pressure.

### DETAILED DESCRIPTION

The present invention provides methods for improving negative pressure wound therapy. The application of negative pressure to a wound bed with a biodegradable biocompatible fibrous scaffold facilitates cell infiltration into the scaffold serving as a 3D structure, as opposed to solely application of the scaffold without any pressure, which shows minimal cell penetration. In vitro studies show that infiltration of immune cells into scaffolds paves the way for their polarization towards the phenotype pertinent to the remodeling stage 2. The cell penetration factor causes less intense immune response of the host body, leading the inflammation to proceed to the remodeling stage.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Improved Negative Pressure Wound Therapy

The present invention relates to improved methods of wound therapy. The methods combine the application of fibrous scaffolds with negative pressure to accelerate and increase the quality of healing wounds, such as chronic/acute skin wounds. The methods enhance the penetration of cells into the scaffold to improve circulation and the recruitment of cells involved in healing.

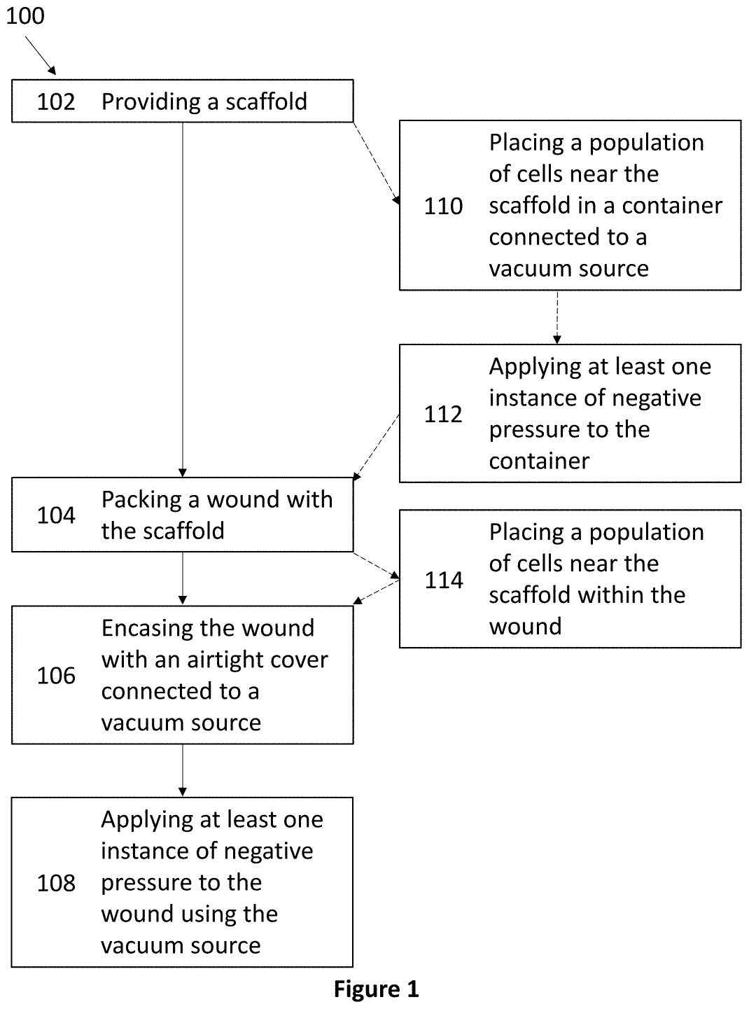

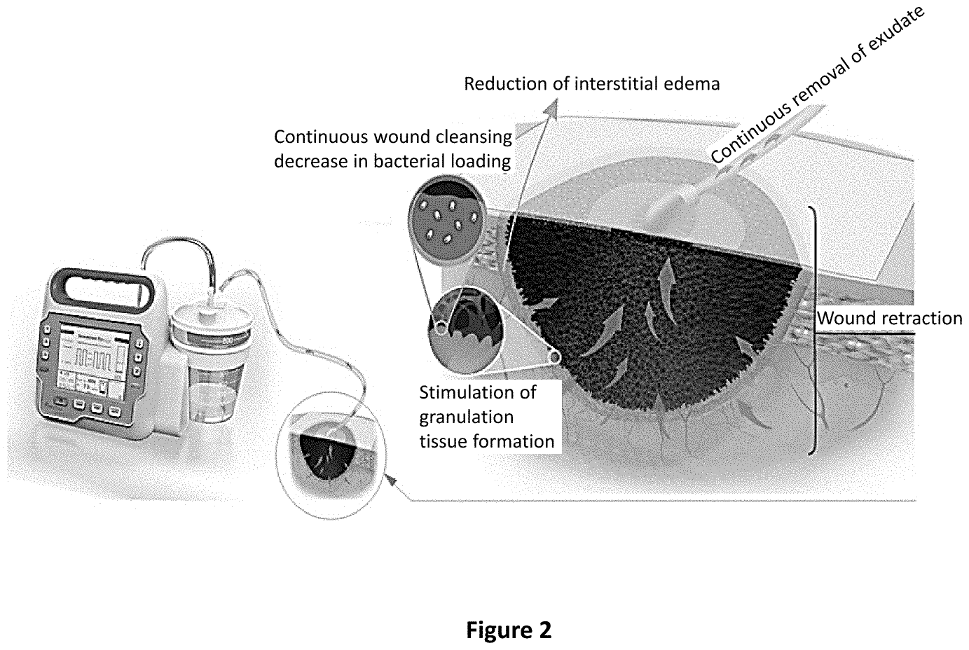

Referring now to FIG. 1, an exemplary method 100 is depicted. Method 100 begins with step 102, wherein a scaffold appropriate for treating a wound is provided. In step 104, the scaffold is packed into a wound. In step 106, the wound is encased with an airtight cover connected to a vacuum source. In step 108, at least one instance of negative pressure is applied to the wound using the vacuum source.

In some embodiments, the scaffold can be pretreated with a population of cells. For example, in some embodiments, method 100 comprises intermediary step 110 of placing a population of cells near the scaffold in a container connected to a vacuum source, and step 112 of applying at least one instance of negative pressure to the container. Intermediary steps 110 and 112 thereby pretreat the scaffold by penetrating the population of cells into the scaffold before placing the scaffold within a wound.

In some embodiments, the scaffold treatment of the wound can be supplemented with a cell treatment. For example, in some embodiments, method 100 comprises intermediary step 114 of placing a population of cells near the scaffold within the wound. Intermediary step 114 thereby provides an additional population of cells near the scaffold in the wound to enhance the negative pressure therapy in successive steps 106 and 108.

The scaffolds described above can be any suitable scaffold that encourages wound healing. In some embodiments, the scaffold is biocompatible. In some embodiments, the scaffold is bioabsorbable. In some embodiments, the scaffold comprises electrospun fibers. Electrospun fibers can be fabricated using any suitable method. For example, electrospun fibers can be fabricated using an electric field in the range of about 5 to about 50 kV. The feed rate of a spinning solution to the spinneret can be in the range of about 0.1 to about 3 mL/hour. The spinneret can be supplemented with one or more additional air jet. The spinning solution can be deposited onto a stationary or a rotating substrate. Persons skilled in the art will understand that a rotating substrate typically involves a mandrel mechanically attached to a motor, often through a drill chuck. In various embodiments, the motor rotates the mandrel at a speed of between about 1 revolution per minute (rpm) to about 40,000 rpm. In an exemplary embodiment, the motor rotation speed is between about 1000 rpm to about 4000 rpm. In another exemplary embodiment, the motor rotation speed of between about 1 rpm to about 300 rpm.

The scaffold fibers can have any suitable diameter, such as between 0.5 m and 5 μm. The scaffold can have any suitable pore size, such as between 1 μm and 10 μm. The scaffold can have any suitable shape, such as a substantially planar sheet or a three dimensional structure, such as a tube or a sphere. In certain embodiments, the scaffold can be trimmed from a larger scaffold to fit the shape and geometry of a wound.

The scaffold can thereby have a thickness suitable for fitting within the wound, such as a thickness that is less than 100 μm or as great as several millimeters.

In certain embodiments, the electrospun fibers of the scaffold can include a polymer. Suitable polymers include but are not limited to: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. Polymers with cationic moieties can also be used, such as poly(allyl amine), poly(ethylene imine), poly(lysine), and poly(arginine). The polymers may have any molecular structure including, but not limited to, linear, branched, graft, block, star, comb, and dendrimer structures.

In certain embodiments, the electrospun fibers of the scaffold can include plant proteins. The plant proteins can be derived from any suitable plant, such as soy protein isolate, wheat gluten, corn zein, pea protein, and the like. A soy protein isolate is a soy material having a protein content of at least 90% soy protein on a moisture free basis. “Isolated soy protein”, as used in the art, has the same meaning as “soy protein isolate” as used herein and as used in the art. A soy protein isolate is formed from soybeans by removing the hull and germ of the soybean from the cotyledon, flaking or grinding the cotyledon and removing oil from the flaked or ground cotyledon, separating the soy protein and carbohydrates of the cotyledon from the cotyledon fiber and lipids, and subsequently separating the soy protein from the carbohydrates. In certain embodiments, the resultant material is washed with ethanol to remove a percentage of isoflavonoids. In one embodiment, the soy-based composition comprises a fibrous material containing soy protein and soy cotyledon fiber. The fibrous material generally comprises a defatted soy protein material and soy cotyledon fiber. The fibrous material is produced by extruding the soy protein material and soy cotyledon fiber. Additional description of soy protein isolate formulations are described in International Application Number PCT/US2016/043388, the contents of which are incorporated herein in its entirety.

In certain embodiments, the scaffolds can include one or more extracellular matrix material and/or blends of naturally occurring extracellular matrix material, including but not limited to collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. The scaffolds can further comprise one or more carbohydrates such as chitin, chitosan, alginic acids, and alginates such as calcium alginate and sodium alginate. These materials may be isolated from plant products, humans or other organisms or cells or synthetically manufactured. Also contemplated are crude extracts of tissue, extracellular matrix material, or extracts of non-natural tissue, alone or in combination. Extracts of biological materials, including but are not limited to cells, tissues, organs, and tumors may also be included.

In various embodiments, the scaffolds can include one or more therapeutics. The therapeutics can be natural or synthetic drugs, including but not limited to: analgesics, anesthetics, antifungals, antibiotics, anti-inflammatories, nonsteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents.

In some embodiments, the scaffolds can further comprise natural peptides, such as glycyl-arginyl-glycyl-aspartyl-serine (GRGDS), arginylglycylaspartic acid (RGD), and amelogenin. In some embodiments, the scaffolds can further comprise proteins, such as chitosan and silk. In some embodiments, the scaffolds can further comprise sucrose, fructose, cellulose, or mannitol. In some embodiments, the scaffolds can further comprise extracellular matrix proteins, such as fibronectin, vitronectin, laminin, collagens, and vixapatin (VP12). In some embodiments, the scaffolds can further comprise disintegrins, such as VLO4. In some embodiments, the scaffolds can further comprise decellularized or demineralized tissue. In some embodiments, the scaffolds can further comprise synthetic peptides, such as emdogain. In some embodiments, the scaffolds can further comprise nutrients, such as bovine serum albumin. In some embodiments, the scaffolds can further comprise vitamins, such as vitamin B2, vitamin Ad, Vitamin D, Vitamin E, and Vitamin K. In some embodiments, the scaffold can further comprise nucleic acids, such as mRNA and DNA. In some embodiments, the scaffolds can further comprise natural or synthetic steroids and hormones, such as dexamethasone, hydrocortisone, estrogens, and its derivatives. In some embodiments, the scaffold can further comprise growth factors, such as fibroblast growth factor (FGF), transforming growth factor beta (TGF-β), and epidermal growth factor (EGF). In some embodiments, the scaffolds can further comprise a delivery vehicle, such as nanoparticles, microparticles, liposomes, viral and non-viral transfection systems.

The population of cells described above can include any cell that contributes to wound healing. For example, in certain embodiments, the wound is a cutaneous wound, muscle wound, diabetic ulcer, burn wound, or surgical opening. Non-limiting examples of suitable cell populations include fibroblasts, keratinocytes, melanocytes, Langerhans cells, myocytes, monocytes, macrophages, and differentiated and undifferentiated stem cells. In other embodiments, the wound is an internal wound, such as to an organ. The cell population can thereby be specific to the wounded organ, such as the liver, kidney, spleen, lung, pancreas, and the like. In some embodiments, the population of cells is at least partially derived from a patient's own tissue. In some embodiments, the population of cells is at least partially derived from another subject within the same species as the patient. In some embodiments, the population of cells is at least partially derived from a mammalian species that is different from the patient. For example the cells may be derived from organs of mammals such as humans, monkeys, dogs, cats, mice, rats, cows, horses, pigs, goats and sheep.

The population of cells may be isolated from a number of sources, including, for example, biopsies from living subjects and whole-organ recovery from cadavers. The population of cells may be isolated using techniques known to those skilled in the art. For example, the tissue may be disaggregated mechanically and/or treated with digestive enzymes and/or chelating agents that weaken the connections between neighboring cells making it possible to disperse the tissue into a suspension of individual cells without appreciable cell breakage. Enzymatic dissociation may be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase and dispase. Mechanical disruption may also be accomplished by a number of methods including, but not limited to, scraping the surface of the tissue, the use of grinders, blenders, sieves, homogenizers, pressure cells, or sonicators.

Once the tissue has been reduced to a suspension of individual cells, the suspension may be fractionated into subpopulations from which the cells elements may be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting.

Cell fractionation may also be desirable, for example, when the donor has diseases such as cancer or metastasis of other tumors to the desired tissue. A cell population may be sorted to separate malignant cells or other tumor cells from normal noncancerous cells. The normal noncancerous cells, isolated from one or more sorting techniques, may then be used for tissue reconstruction.

Isolated cells may be cultured in vitro to increase the number of cells available for seeding the biomimetic scaffold. The use of allogenic cells, and more preferably autologous cells, is preferred to prevent tissue rejection. However, if an immunological response does occur in the subject after implantation of the artificial organ, the subject may be treated with immunosuppressive agents such as cyclosporin or FK506 to reduce the likelihood of rejection. In certain embodiments, chimeric cells, or cells from a transgenic animal, may be seeded onto the biocompatible scaffold.

Isolated cells may be transfected prior to coating with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chances of rejection by the host. In addition, transfection could also be used for gene delivery.

As described above, at least one instance of negative pressure is applied to the scaffold and the wound at various steps to improve cell penetration and wound healing. The negative pressure can be any suitable pressure, such as in the range of about 1 to 10 psi. The duration of negative pressure can be for any suitable time, such as between about 5 and 50 seconds. In some embodiments, the negative pressure is applied continuously for the duration of a treatment session. In some embodiments, the negative pressure is applied in a pattern, such as a pulsatile pattern. In some embodiments, the negative pressure treatment is repeated several times over the course of treatment, wherein the at least one instance of negative pressure is applied at least once per hour, at least once per day, or at least once per week.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, make and utilize the compounds of the present invention and practice the claimed methods. The following working examples therefore, specifically point out the exemplary embodiments of the present invention, and are not to be construed as limiting in any way the remainder of the disclosure.

### Example 1: Negative Pressure Improves Cell Penetration into Electrospun Fibrous Scaffolds

Described below is a method to improve the pace and quality of healing in skin wounds. Briefly, the methodology consists of application of sub-atmospheric pressure to wound beds while a fibrous electrospun scaffold is applied to the wound bed. Specifically, electroprocessed nano/micro-fibrous scaffolds made from synthetic or natural polymers are applied. In one embodiment, the main component of the scaffold is purified soy protein isolate (SPI), a biocompatible material that has been shown to allow attachment, spreading and proliferation of cells in vitro (Lin, L et al., 2013, Journal of tissue engineering and regenerative medicine 7(12):994-1008).

The materials and methods are now described.

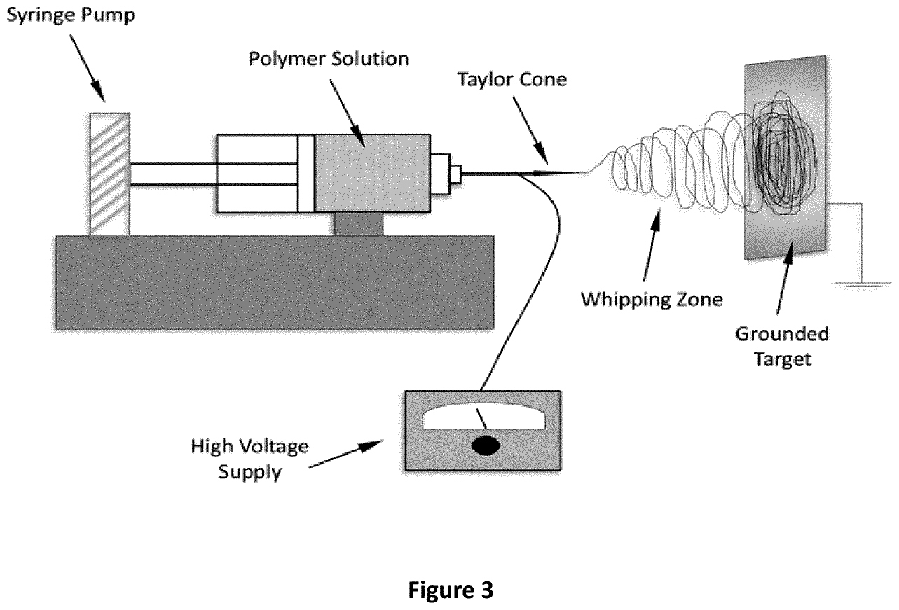

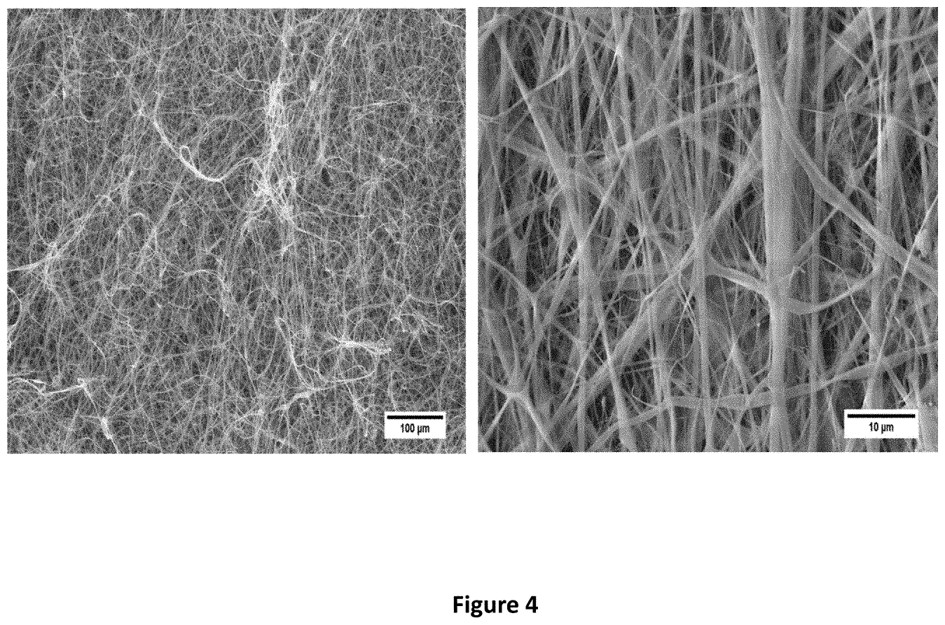

7% (w/v) of purified soy protein isolate (SPI) plus 0.05% (w/v) polyethylene oxide (PEO) were dissolved in 1,1,1,3,3,3-Hexafluoro-2-propanol (HFP) and electrospun onto an aluminum target. A spinning system was set up with the SPI spinnerets connected to a high voltage electrostatic field. A vertical stationary flat aluminum target was used to collect the fibers. The nozzle was arranged perpendicular to the target (FIG. 3). This setup generates a fibrous scaffold containing small fibers of SPI (1.15±0.4 μm) (FIG. 4). The mechanical properties of the scaffolds were investigated using a Bose Electroforce materials testing system (MTS) with a load cell of 10N.

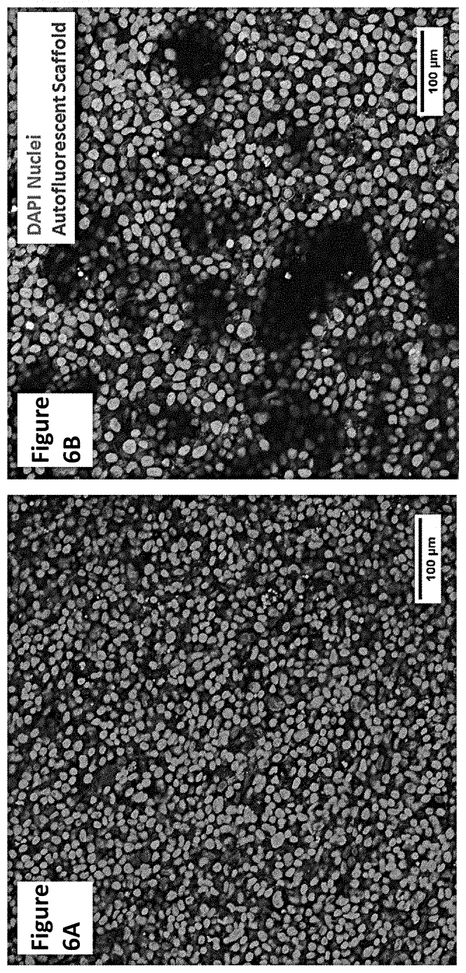

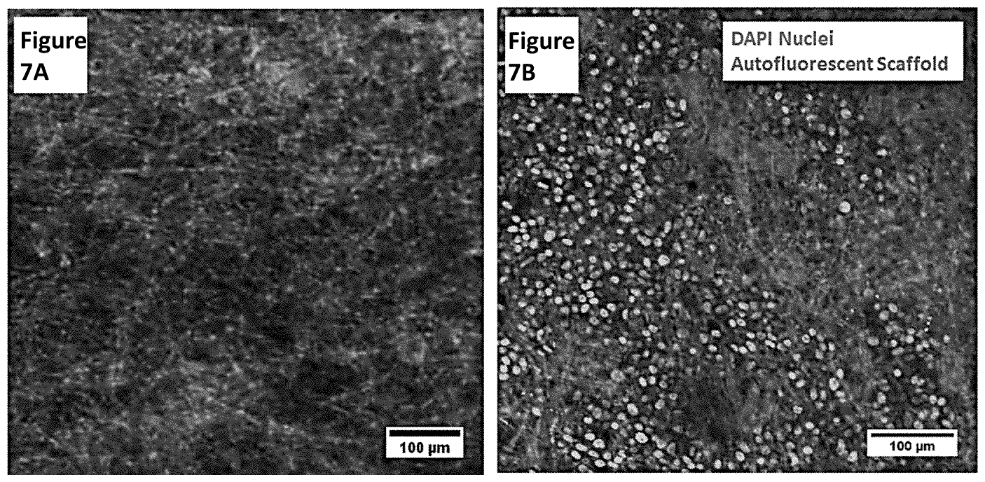

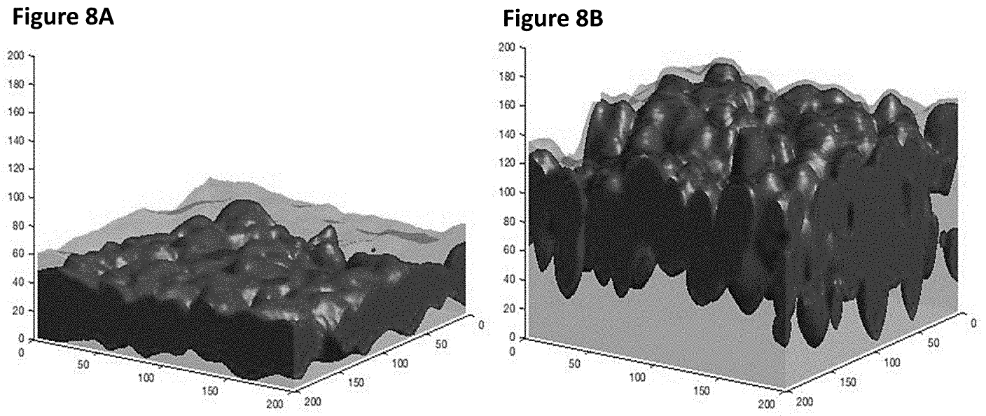

For the cell seeding experiments, the scaffolds were placed and sealed inside Whatman plastic filter holders. HaCaT cells, a human keratinocyte cell line, were seeded at a density of 100,000 cells/cm² directly on top of the scaffolds. A house vacuum of 4 psi was applied for 10 seconds to the bottom side of the filter holder, no pressure was applied to the control group. FIG. 6A and FIG. 6B shows surface view of scaffolds under standard condition and with negative pressure. Three days post cell seeding, samples were retrieved, stained with DAPI (4′,6-diamidino-2-phenylindole, Life Technologies), and visualized using a FV1200 Olympus laser scanning microscope. Cell penetration was quantified by FIJI software (ImageJ 1.50 g). Cellular penetration into the mid-layer (70 μm in depth) of electrospun SPI scaffolds both under standard condition and with negative pressure is shown in FIG. 7A and FIG. 7B. In order to better understand the organization of cells throughout the thickness of the scaffold (140 μm), a MATLAB® manuscript was developed with the resultant 3D reconstructed image shown in FIG. 8A and FIG. 8B.

The results are now described.

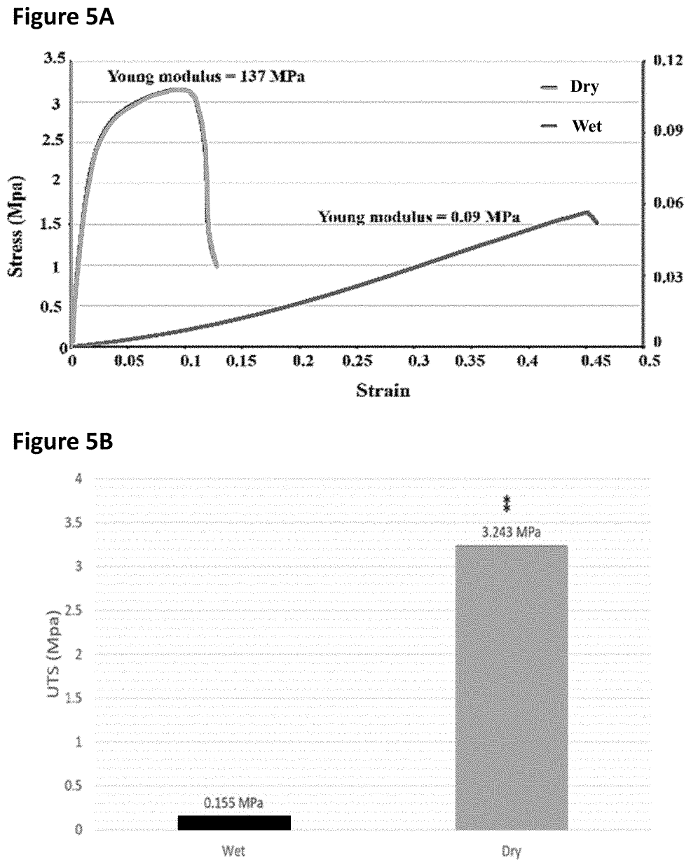

Comparison of the mechanical properties of the scaffolds in dry and wet conditions showed a significant increase in the deformability of the wet samples, concomitant with a drastic decrease in both ultimate tensile stress and young modulus, respectively, from 3.15 MPa and 137 MPa in dry condition to 0.09 MPa and 0.06 MPa (FIG. 5A and FIG. 5B). Seeding the cells under negative pressure enhanced cell penetration into our densely packed electrospun scaffolds with an inherent pore diameter of 4.1±1.3 μm (FIG. 6A and FIG. 6B). Application of negative pressure promoted infiltration of HaCaT cells into deeper layers of the scaffold, as opposed to almost zero penetration in the control group (FIG. 7A and FIG. 7B).

The results demonstrate that application of negative pressure enhances cellular penetration into dense electrospun scaffolds with innate pores smaller than the diameters of the cells in suspension. This methodology has the potential for improving the regenerative capacity of both acellular and pre-seeded fibrous scaffolds for numerous applications in regenerative engineering, including in cutaneous wound healing

The ultimate goal of this study is to employ a portable bedside NPWT system in conjunction with bioengineered fibrous scaffolds for facilitating and enhancing healing in cutaneous wounds. The long-term goal of the study is to explore how application of vacuum can accelerate and facilitate penetration of skin cells, e.g. of immune cells, specifically of macrophages, into deeper layers of scaffold. The study will demonstrate that the early and enhanced penetration of macrophages into the scaffolds will favor the macrophages' phenotype switch (polarization) towards the tissue remodeling M2 phenotype 2-4 and initiate and accelerate regenerative wound-healing.

Additional experiments will optimize the negative pressure regimen (amplitude/duration) for maximal scaffold penetration without affecting cell viability in vitro. Further studies will also investigate whether the penetration induced by vacuum causes any significant phenotype switch in macrophages. The studies will assess the phenotypic switch, a.k.a. “polarization” using human monocyte cell line THP-1. Additional studies will test the functionality of the newly developed system in a full thickness cutaneous in vivo study, wherein phenotypes of macrophages targeting scaffolds in situ will be assessed at different time points (1-28 days). This investigation will provide a ratio of anti-inflammatory (M2) to pro-inflammatory (M1) phenotype, which will be correlated with the stage of healing. In addition, the investigation will also evaluate the degree of re-epithelization of the tissue, the extent of angiogenesis, and the restoration of the skin appendages, such as hair follicles and sweat glands.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of enhancing wound healing, comprising the steps of:
providing a scaffold;
packing a wound with the scaffold;
encasing the wound with an airtight cover connected to a vacuum source; and
applying at least one instance of negative pressure to the wound using the vacuum source.

2. The method of claim 1, wherein the provided scaffold is pretreated by the steps of:
placing a population of cells near the scaffold in a container connected to a vacuum source; and
applying at least one instance of negative pressure to the container.

3. The method of claim 1, wherein a population of cells is placed near the scaffold packed into the wound.

4. The method of claim 1, wherein the provided scaffold comprises electrospun soy protein isolate.

5. The method of claim 1, wherein the provided scaffold is electrospun from a solution comprising 7% (w/v) purified soy protein isolate (SPI) and 0.05% (w/v) polyethylene oxide (PEO) dissolved in 1,1,1,3,3,3-Hexafluoro-2-propanol (HFP).

6. The method of claim 1, wherein the provided scaffold comprises fibers having a diameter of between 0.5 and 5 μm.

7. The method of claim 2, wherein the population of cells comprises at least one cell selected from the group consisting of: fibroblasts, keratinocytes, melanocytes, monocytes, and macrophages.

8. The method of claim 1, wherein the wound is a cutaneous wound, a muscle wound, a diabetic ulcer, a burn wound, or a surgical opening.

9. The method of claim 1, wherein the at least one instance of negative pressure is applied using a negative pressure wound therapy system.

10. The method of claim 1, wherein the at least one instance of negative pressure applied is between 1 and 10 psi.

11. The method of claim 1, wherein the at least one instance of negative pressure is applied for a duration of between 5 and 50 seconds.

12. The method of claim 1, wherein the at least one instance of negative pressure is applied continuously.

13. The method of claim 1, wherein the at least one instance of negative pressure is applied in a pulsatile pattern.
